(19) United States
(12) Patent Application Publication
(10) Pub. No.: US 2016/0331271 A1
(43) Pub. Date: Nov. 17, 2016

(54) A MANUAL RESUSCITATOR AND CAPNOGRAPH ASSEMBLY

(71) Applicant: SENSEBREATH AB, Sorunda (SE)

(72) Inventor: Andras GEDEON, Stockholm (SE)

(73) Assignee: SENSEBREATH AB, Sorunda (SE)

(21) Appl. No.: 15/112,346

(22) PCT Filed: Jan. 19, 2015
(86) PCT No.: PCT/SE2015/050045

§ 371 Date: Jul. 18, 2016

Related U.S. Application Data
(60) Provisional application No. 61/931,728, filed on Jan. 27, 2014.

(51) Int. Cl. A61B 5/08; A61M 16/20; A61M 16/08; A61M 16/10; A61B 5/00; A61M 16/00

(52) U.S. Cl. CPC A61B 5/082; A61B 5/6898; A61B 5/742; A61B 5/7271; A61B 5/0077; A61M 16/0084; A61M 16/085; A61M 16/1045; A61B 5/0816; A61M 16/20

(57) ABSTRACT

A manual resuscitator assembly for measuring the carbon dioxide (CO2) concentration in breathing gas of a person being ventilated, includes:
a selective colorimetric CO2 detector having a detector surface adapted to change color rapidly and reversibly with CO2 concentration,
a detector holding part for receiving and attaching the colorimetric detector,
a manual resuscitator including a bi-directional gas conduit between an expiratory and an inspiratory part of a breathing circuit, the conduit configured such that a clinically predetermined fraction of breathing gas enters the holding part and contacts the surface during ventilation,
a docking part for receiving and attaching a mobile unit, including image capturing, processing and a display elements, the processing element adapted to execute an application program to measure CO2 concentration changes in the breathing gas by identifying changes in the optical property of the detector surface captured by the image capturing element.

### FIELD OF THE INVENTION

The present invention relates to a modified manual resuscitator that incorporates a colorimetric capnograph for monitoring exhaled carbon dioxide (CO2) concentration and displaying capnograms. More particularly, the invention relates to an assembly that is compact, inexpensive and user-friendly and is particularly advantageous during CPR (Cardio Pulmonary Resuscitation).

### BACKGROUND OF THE INVENTION

Infrared absorption (IR) is the state of the art method for measuring exhaled carbon dioxide (CO2) concentration and instruments based on this technology (IR capnographs) have been in clinical use for more than 30 years. They are used routinely for monitoring in the operating theatre and in postoperative care. The exhaled CO2 concentration curve (the so called capnogram) has also been used together with respirator treatment and for more sophisticated diagnostics for instance of lung function.

The IR technology is inherently complex and expensive with advanced optical and electronic components. In recent times compact models have been developed that are portable and can also be used in emergency situations. However, to obtain sufficient absorption for an acceptable signal to noise ratio it is necessary for the IR beam to pass through a significant volume of the gas to be analyzed. This sets a limit on how small an IR detector can be.

A different method of detecting CO2 is based on durable, rapid and reversible colorimetric detectors that change color with the concentration of the CO2. Such a detector is described in A Gedeon, P Krill and C Mebius: A new colorimetric breath indicator (Colibri), Anaesthesia 1994 (49) 798.

The colorimetric method involves a color change of the surface of a thin membrane. This means that only a very small volume of gas needs to pass over the surface, to produce satisfactory conditions for CO2 detection.

Colorimetric detectors of CO2 are less costly and can be part of devices for single patient use. They serve today as semi quantitative visual indicators for verifying proper tracheal tube placement (AirLife CO2 detectors, CareFusion US). The color change of the detector produced by the CO2 issuing from the patient confirms that the trachea and not (accidentally) the esophagus has been intubated.

Recently it has been shown, U.S. patent application No. 61/870,858, that it is possible to obtain quantitative CO2 concentration data and capnograms of good quality using colorimetric detectors and a mobile unit, comprising an image capturing means, a processing means and a display, such as for instance a standard smartphone. The present invention teaches how the advantages of these technologies can be combined and integrated into manual resuscitators.

Measuring expired CO2 concentration is most desirable in connection with manual ventilation in general and during CPR in particular because it helps assess the effectiveness of ventilation and also indicates the circulatory status of the patient. The Return Of Spontaneous Circulation (ROSC), the ultimate goal of CPR, is immediately recognized by the return of CO2 in the exhaled gas. The so called end-tidal CO2 (etCO2) concentration (the high value at the end of the expired breath) is commonly measured but since the significance of this value is difficult to assess during the varying respiratory and circulatory conditions encountered during CPR, it is necessary to observe the true capnogram as well. This has been recognized in the American Heart Association (AHA) guidelines for CPR procedures that recommend the monitoring of exhaled CO2 by capnograms. (Circulation 2010; 122: p 640-933 American Heart Association Guidelines for Cardiopulmonary Resuscitation and Emergency Cardiovascular Care Science).

There are two types of IR instrumentation on the market. One type samples breathing gas from the patient using a pump and can display both etCO2 values and capnograms (MaCO₂ module from Nonin Medical Inc., US). These instruments are expensive, need a power supply and are not well suited for ambulatory use. There are also compact battery operated units connected directly to the breathing tube of the patient (EMMA Mainstream capnometer, Masimo Sweden AB, Sweden) but these devices are also expensive and they are susceptible to failure due to mucus or other body fluids of the patient affecting the measuring cell and thereby the transmission of the IR beam. Furthermore, the immediate proximity to the patient limits available space, so that these instruments can only display etCO2 values and not true capnograms.

Thus, there is a strong need for a low cost clinically robust, easy to use device that can show true capnograms as well as etCO2 values and can be an integral part of a manual resuscitator.

The present invention addresses all the above requirements. In particular, the object of the present invention is to provide a most convenient, simple and inexpensive way to display a capnogram during manual ventilation and especially during CPR.

### SUMMARY OF THE INVENTION

The above mentioned objective is achieved by the present invention according to the independent claim.

The present invention is based upon the well-established technique of colorimetric CO2 sensing. More specifically, a thin membrane is provided with a smooth continuous coating of chemicals so that its surface changes color selectively for CO2 and in a fast, reversible way meaning that if the membrane is for instance blue in room air and yellow at about 5% CO2 then it will change from blue to yellowish during a typical exhalation and then return to the initial blue color during inspiration. It will thus cycle between blue and yellowish when exposed to breathing gas.

Such a detector is integrated with the resuscitator through an attachment that establishes a gas conduit between the inspiratory part and the expiratory part of the breathing circuit block of the resuscitator. For each breath a very small bidirectional flow of gas will pass through this conduit, one direction during inspiration and the opposite direction during expiration. The CO2 detector is placed in the conduit in such a way that its color is observable from outside. Since the colorimetric CO2 detector operates using a surface effect, the amount of gas passing the conduit for each breath can be strongly restricted to a predetermined fraction, typically only about 1-3%, of the volume of a breath and so the shunt flow in the conduit is negligible compared to the ventilation of the patient. A device, such as a standard smartphone, is then connected to the attachment so that it can image the detector, encode its colors, calculate the CO2 concentration and display the capnogram and preferably also the etCO2 value and/or the respiratory rate.

### SHORT DESCRIPTION OF THE APPENDED DRAWINGS

FIG. 1 is a schematic cross-sectional view of a standard self-inflating manual resuscitator.

FIG. 2 illustrates in cross section the attachment with the CO2 detector in the gas conduit connecting the inspiratory part and the expiratory part of the breathing circuit block of the resuscitator. The view is as seen by the person holding the resuscitator when looking in the direction of the patient.

FIG. 3 illustrates the attachment from a side view with and without a protective cover of the CO2 sensor. The place designated for a mobile unit is indicated.

FIG. 4a illustrates a mobile unit comprising an image capturing means, a processing means and a display with a holder for docking with the attachment on the resuscitator.

FIG. 4b illustrates the mobile unit in operation after docking to the modified resuscitator.

### DETAILED DESCRIPTION OF PREFERRED EMBODIMENTS OF THE INVENTION

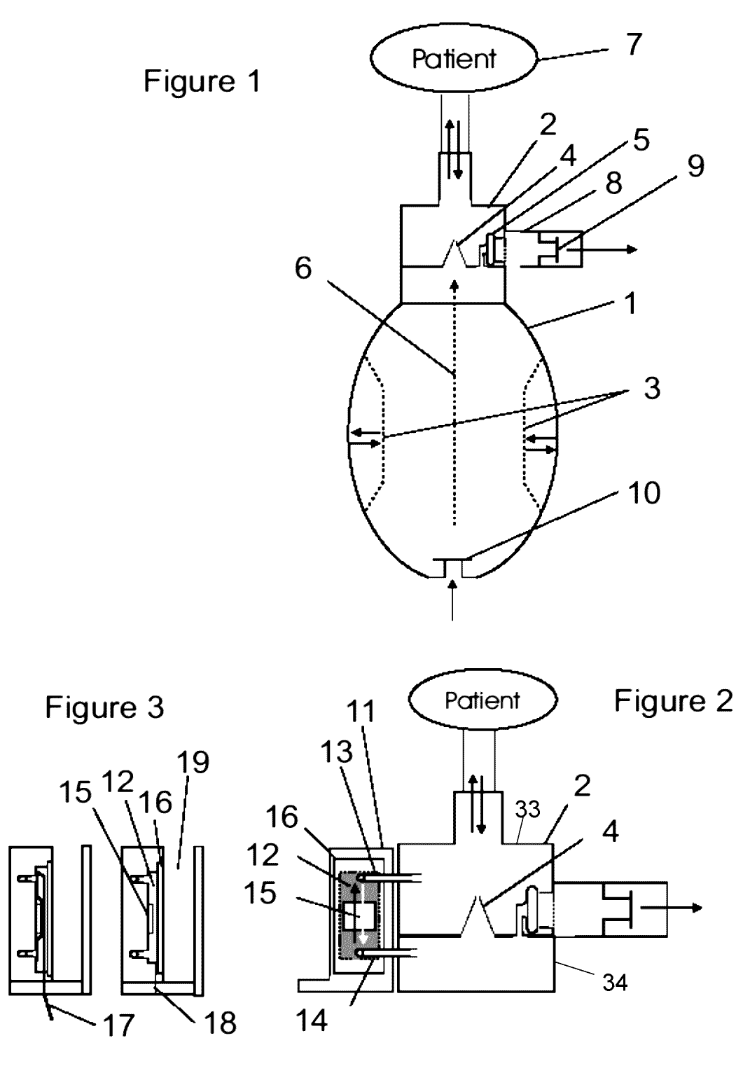

First with references to FIG. 1 the characteristics and operation of a typical self-inflating resuscitator is shown. The main components of the device are the self-inflating rubber bag 1 and the breathing circuit block 2 providing the connection to the patient. Gas is delivered to the patient (inspiration) when the bag is squeezed 3 (illustrated by the inwards-directed arrows) creating a positive pressure in the bag. This opens a so called fishmouth unidirectional valve 4 and closes the expiratory port for instance with a mushroom valve 5 and directs the gas 6 from the bag to the patient 7. Expiration starts when the bag is let go 3 and a negative pressure is generated in the bag by the self-expansion 3 (illustrated by the outwards-directed arrows). Now the fishmouth valve 4 closes and the mushroom valve 5 opens allowing the expired gas to exit at the resuscitator at port 8 where a one-way valve 9 prevents gas from entering during inspiration. The bag is eventually refilled by sucking in gas from the ambient (with or without added oxygen) through a one-way valve 10 at the bottom of the bag.

FIG. 2 shows the attachment 11 to the breathing circuit block 2 according to one preferred embodiment of the present invention with a gas conduit 12 established between ports 13 and 14 that respectively is connected to an expiratory part 33 (grey in the figure) and an inspiratory part 34 of the breathing circuit block. The expiratory part 33 is separated from the inspiratory part 34 by a wall where the one-way valve 4 is arranged such that during inspiration air flows from the inspiratory part via the valve into the expiratory part and to the patient.

The gas conduit between the expiratory and the inspiratory part of the resuscitator is preferably realised by openings in the walls of the expiratory and inspiratory parts, and by gas conduits, e.g. flexible or rigid tubes, connecting the openings to the detector holding part.

The gas in the conduit will move from the bag towards the patient during inspiration while during expiration the positive pressure on the patient side of the valve 4 and the negative pressure in the bag generated by the expansion 3 will draw expiratory gas through the conduit.

A CO2 detector 15 is placed in the detector holder part of the conduit 12 between the two ports 13 and 14. The cross section of the conduit at the detector is preferably square in shape with the wall 16 facing the detector 15 made of a transparent material such as Polymethylmethacrylate (plexiglass) or Polycarbonate (macrolon). As an example, if the membrane of the detector has dimensions 5×5×0.1 mm and the cross section of the gas conduit at the detector is 0.7×10 mm then the volume of the gas around the detector is only <0.03 cc. The total volume of the gas conduit may preferably be typically 0.2-0.5 cc. Since the volume of a tidal breath is normally in the range 15-1000 cc (infant to adult) it is apparent that the flow needed to flush the conduit will be a predetermined fraction, typically less than 1-3%, of the total ventilation, which produces no clinically observable effects. Because the flow is very low and bidirectional and because of the location of the conduit is away from the main breathing gas passage, interference from condensation or body fluids is highly unlikely.

FIG. 3 shows the attachment 11 in a side view. Initially when in storage, the detector may be covered with a seal 17 (see the left part of FIG. 3), as an example a 0.07-0.15 mm thick aluminium/plastic foil, so that it is protected from prolonged exposure to ambient air. The seal also protects the sensor from bleaching by prolonged exposure to UV radiation. The seal is removed prior to use by pulling it through a tight slit 18 placed on the side of the gas conduit near port 14, so that it is downstream of the detector 15 when it is exposed to the expiratory flow. The ready-to-use attachment 11 is illustrated by the right part of FIG. 3.

The attachment 11 is configured to receive a mobile unit comprising an image capturing means, a processing means and a display. More specifically, the attachment 11 comprises attachment walls forming a hollow space 19 such that the mobile unit may be firmly held in a predetermined position in relation to the detector 15.

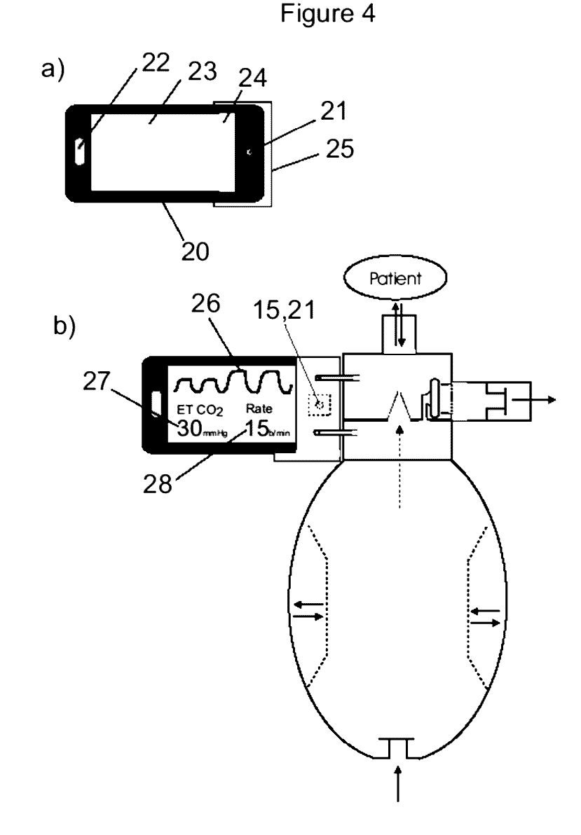

FIG. 4a shows such a mobile unit in the form of a standard smartphone 20. The camera 21 and the control 22 of the mobile unit both face the operator of the resuscitator when the unit is received by the attachment 11. So does the display 23, the main part of which is used for presenting the CO2 concentration data and a smaller part 24 next to the camera is preferably used to illuminate the detector. The unit may preferably be fitted with a holder 25 that enables easy connection to the attachment on the resuscitator.

FIG. 4b illustrates the entire assembly provided with the imaging system aligned with the CO2 sensor. In operation the display can present in direct view of the operator capnograms 26, etCO2 values 27 and the respiratory rate 28. At the start of measurements the color of the colorimetric detector is determined by the mobile unit in the absence of CO2. If this initial state is outside the allowed range the unit may compensate for this deviation either by correcting the preprogrammed relation between the color change and the CO2 concentration or by adjusting the color of the illuminating light. If the detector is entirely out of the acceptable range the unit can simply post a message of detector rejection on the display.

From the above description it is obvious that the same functions may be obtained in many different ways. The attachment 11 may be a permanent part of the resuscitator but could also be a separate entity. The attachment could also be integrated with the holder 25 of the mobile unit and together they could be connected to the resuscitator.

In order to improve the user-friendliness of the assembly the attachment 11, in one embodiment, is configured such that the mobile unit, and thus the display, is movable in relation to the breathing circuit block. Thereby the display may be put in a position such that the user easily may operate the mobile unit and monitor the display. This may be achieved by allowing the attachment to rotate a predetermined angle (e.g. 0-45 degrees) around an axis defined by one of the tubes connected to the openings in the breathing circuit block, thus one tube is then rigid and the other is flexible. In a further embodiment a separate rotation and fastening part is provided between the tube connections defining a rotational axis. In this embodiment both tube connections are flexible.

The gas conduit 12 with the CO2 detector may be a permanent part of the attachment but could also be separate such that a detector holder unit would be introduced into the attachment prior to use. It is also possible to introduce a bacterial filter in the gas conduit at the expiratory port 13 if bacterial protection of the attachment is desired. The mobile unit is at all times separated from the breathing gas of the patient.

Furthermore, it is beneficial for the function of the detector that the conditioning of the gas from the rubber bag and the gas from the patient is made as similar as possible. In order to minimize the difference of the gas with regard to heat and moisture a heat and moisture exchanging material may be introduced into the gas conduit. Preferably this material is in the form of a porous structure presenting little resistance to the gas flow but exposing a large surface area to the gas passing through the conduit. As one example polyurethane foam of suitable porosity (EMW filtertechnik GmbH) is used.

Also, the mobile unit may have many different shapes and functions. It could encode the colors of the sensor but also any other optical property (such as the reflectance or the transmittance) that changes with the CO2 concentration. Because of its convenient proximity to the operator it could not only provide visual information on the display as shown in FIG. 4b but also audible information such as alarms and/or instructions. It could also store the data for later evaluation or send it in various technically established ways to a remote receiving entity. In addition, the mobile unit may have means to illuminate the patient for ease of observation of particular use under difficult lightening conditions.

Finally, the resuscitator may be designed in many ways with different valve systems, bag types and ways of refilling the bag. The basic requirement of the present invention to establish a gas conduit between the inspiratory and expiratory parts may be realized in different ways both with fixed arrangements and with flexible components.

1-21. (canceled)

22. A manual resuscitator assembly configured to allow measurement of the carbon dioxide (CO2) concentration in breathing gas of a person being ventilated, the assembly comprises:
a selective colorimetric CO2 detector provided with a detector surface adapted to change color rapidly and reversibly with the concentration of CO2, when exposed to CO2,
a detector holding part adapted to receive said colorimetric detector and attach said detector,
a manual resuscitator including a self-inflating rubber bag and a breathing circuit block for providing connection to the patient, and further including a bi-directional gas conduit between an expiratory part and an inspiratory part of said breathing circuit block of the resuscitator for providing a shunt flow in the gas conduit, wherein the gas conduit between the expiratory and the inspiratory parts of the resuscitator is realised by openings in the walls of the expiratory and inspiratory parts, and by flexible or rigid gas conduits connecting said openings to said detector holding part, and wherein said gas conduit being configured such that a very small predetermined fraction of the breathing gas enters said detector holding part and contacts said detector surface during ventilation,
a docking part for receiving and attaching a mobile unit, which comprises an image capturing means, a processing means and a display, wherein the docking part is configured to position said image capturing means in a fixed relation to said colorimetric detector, such that said image capturing means may capture an optical property of said detector surface, and wherein said processing means is adapted to execute an application program adapted to perform a measurement of CO2 concentration changes in the breathing gas by identifying changes in the optical property of said detector surface captured by said image capturing means.

23. The assembly according to claim 22, wherein said very small predetermined fraction of the breathing gas is less than 3%.

24. The assembly according to claim 22, wherein said gas conduit is provided with a heat and moisture exchanging material, and that the material has a porous structure presenting little resistance to the gas flow but exposing a large surface area to the gas passing through the conduit.

25. The assembly according to claim 22, wherein said detector holding part includes a detector surface protecting means adapted to protect the surface from ambient air in an airtight fashion prior to use of the assembly.

26. The assembly according to claim 25, wherein said detector surface protecting means is adapted to be moved to a non-protection position, such that said detector surface is available for measurements.

27. The assembly according to claim 26, wherein said detector surface protecting means is adapted to be removed through an opening to the ambient where the opening is located downstream relative to the detector considered during an expiratory phase of the breathing.

28. The assembly according to claim 22, wherein said mobile unit is a smartphone.

29. The assembly according to claim 22, wherein said image capturing means is a camera unit.

30. The assembly according to claim 22, wherein said image capturing means comprises illumination means at least one of which is adapted to illuminate said detector surface during measurement.

31. The assembly according to claim 30, wherein the color of the illumination is chosen in relation to the optical characteristics of the detector surface in the absence of CO2.

32. The assembly according to claim 30, wherein said illumination means is at least a part of the display of the assembly.

33. The assembly according to claim 22, wherein during a measurement session a predetermined number of successive images are taken, preferably at least 4-5 images per second, by said image capturing means of at least a part of said detector surface.

34. The assembly according to claim 22, wherein said processing means is adapted to calculate and display capnograms at said display.

35. The assembly according to claim 34, wherein said processing means in addition is adapted to calculate and display end tidal CO2 values and respiratory rates at said display.

36. The assembly according to claim 22, wherein said processing means is adapted to store at least one characteristic optical property of the detector surface, such as the color, or the reflectance.

37. The assembly according to claim 22, wherein said processing means is adapted to store at least one relationship between said characteristic optical property of the detector surface and the corresponding concentrations of CO2.

38. The assembly according to claim 22, wherein said mobile unit includes an audio part wherein auditory alarms and/or instructions are generated to guide the user.

39. The assembly according to claim 22, wherein said measurement session includes checking of the detector quality.

40. The assembly according to claim 39, wherein said detector quality is checked at start of use by a comparison of the optical characteristics of the detector to a reference in the absence of CO2.

41. The assembly according to claim 22, wherein said CO2 detector comprises a porous material containing in its pores a phase transfer agent and a pH sensitive color indicator.

42. The assembly according to claim 41, wherein the phase transfer agent is tetraoctyammoniumhydroxide and the pH sensitive color indicator is thymol blue.
